(19) United States
(12) Patent Application Publication
(10) Pub. No.: US 2009/0118779 A1
(43) Pub. Date: May 7, 2009

(54) Modular System For A Wireless Implantable Device

(76) Inventors: Nader Najafi, Ann Arbor, MI (US); Sonbol Massoud-Ansari, El Dorado Hills, CA (US)

Correspondence Address: JELIC PATENT SERVICES, LLC, 3135 S. STATE ST., SUITE 350-R, ANN ARBOR, MI, 48108, US

(21) Appl. No.: 12/265,827

(22) Filed: Nov. 6, 2008

Related U.S. Application Data
(60) Provisional application No. 61/002,001, filed on Nov. 7, 2007.

(51) Int. Cl. A61N 1/00; A61B 5/00

(52) U.S. Cl. 607/2; 600/300

(57) ABSTRACT

A modular platform for wireless implantable devices. The platform enables different functional units which either measure or affect a parameter of the patient's body to be interchanged. The modularity of the platform also enables the functional unit to be remote from the remainder of the implantable device. Finally, the modularity of the platform enables different types of power sources to supply power to the wireless implantable device.

### CROSS REFERENCE TO RELATED APPLICATIONS

This application claims the benefit of U.S. Provisional Application 61/002,001 filed Nov. 7, 2007. The content of this prior patent application is incorporated by reference.

### BACKGROUND OF THE INVENTION

The present invention relates to wireless implantable devices designed to measure or affect a parameter of a patient's body. Numerous wireless implantable devices are currently available which can measure a parameter of the patient's body. An example is a heart pressure monitor. In addition, there currently are devices designed to affect a parameter of the patient's body.

### BRIEF SUMMARY OF THE INVENTION

The present invention provides a modular platform for wireless implantable devices. The modularity of the invention allows different functional elements to be substituted in the modular platform. The functional elements may either be sensing elements or actuating elements. An external readout unit will wirelessly communicate with the modular platform and may wirelessly provide power to the modular platform.

The modular platform will also enable the functional elements to either be physically integrated with the platform or attached separately. If the functional element is physically integrated, it will share the same housing as the rest of the platform. If the functional element is attached separately, it will be connected to the remainder of the platform electrically and/or mechanically using methods known in the art.

Finally, the modular platform will allow for different sources of power. One possible power source is a battery. The battery may either be chargeable or non-chargeable. A second possible power source is a magnetic telemetry unit, which is capable of receiving transmitted power from the external readout unit.

### BRIEF DESCRIPTION OF THE DRAWINGS

FIG. 1 is a schematic of a standalone wireless implantable platform with components placed inside a housing.

FIG. 2 is a schematic of a standalone wireless implantable platform with components placed inside a substrate.

FIG. 3 is a schematic of a wireless implantable platform with multiple subassemblies.

### DETAILED DESCRIPTION OF THE INVENTION

This disclosure describes a novel flexible platform for small, wireless, implantable devices. The platform allows the rapid realization of different functionalities into wireless, implantable devices. These functionalities include but not limited to sensing (e.g., pressure, flow, cardiac signals, brain signals, etc.), actuating (stimulating, cardiac stimulating, brain stimulating, drug delivery, shock, etc.), and structural functions (e.g., anchor, attachment, enhanced strength, etc.).

In a preferred embodiment, the system includes an implantable unit and a companion external unit. The implantable unit is inserted into an area of interest and the external unit communicates with and/or provides tele-powering to the implantable unit. The external unit may include any combination of an electronic component, antennas, coils, reference devices, software interfaces, etc., based on the intended use of the wireless platform. The implantable part of the wireless platform may include any combination of one or more of the following parts: an interface for attachment of a device with specific functionality, an electronic component, inductor coil, core (e.g., ferrite), battery or charge storing device, and housing. Other components can also be included depending on the intended use of the wireless platform.

Different functionalities can be incorporated into the platform by simply attaching a new component that represents the new functionality (for example a pressure sensor to measure pressure) to the platform. The platform may have (but not necessarily) an electronic component which can be modified (e.g., custom designed or reprogrammed) in order to work with the attached new component. This modular approach allows most of the wireless implantable platform to be shared by different applications. The platform may offer different functions including but not limited to one or more of: tele-powering, power filtering, power management, tele communication (receiving, transmitting or both), conversion of input signals (e.g., sensor output) into electrical signals to be transmitted, receiving commands from the external unit and execute them, send ID information to the external unit, offer anchoring functions, offer delivery (e.g., catheter delivery) options, provide a biocompatible external body, etc.

In one preferred embodiment, the disclosed wireless implantable platform is a miniature stand alone system. The platform will contain one or more interface available such that one or more devices with specific functionalities can be attached via this interface to the wireless implantable platform. These interfaces may include but are not limited to electrical contacts, fluid contacts/interface, optical interfaces, magnetic interface, capacitive interface, or a combination thereof. The interfaces may be either fabricated as part of, or placed on a substrate, such that there will be connections from the interface to the remaining components of the implantable system. The interfaces may be two-dimensional (i.e. pads, electric contacts, etc.), or three-dimensional (i.e. tubes, etc.), or any combination or shape that is useful for the specific application. The substrate on which the interfaces are placed or fabricated on, can be either rigid or flexible, or it can be partly rigid and partly flexible (for example rigid-flex substrates, where part of the substrate is rigid and the other part is flexible). In the case of flexible substrates, various polymers, Parylene, silicone, or other biocompatible flexible material may be used. In the case of rigid substrates, glass, silicon, ceramics, carbides, alloys, metals, hard polymers, Teflon, are some examples of substrate materials, although other types of materials can also be used for the substrate. In the case of rigid-flex substrates, the rigid and flexible parts may be made from dissimilar material. The interfaces would usually be on one side of the substrate, and feedthroughs (preferably hermetic, but not necessary, based on the application) will be used to connect the interfaces to the other components of the system such as such as an electronic component, inductor coil, charge storing device, batteries, or any other kinds of components (based on the application). The inductor coil can be made using any method known in the art, such as winding a conductor around a ferrite core (air cores are possible as well), depositing (electroplating, sputtering, evaporating, screen printing, etc), a conductive coil (preferably made from a highly conductive metal such as silver, copper, gold, etc.), on a substrate (rigid or flexible), or any other method known to those skilled in the art. The inductor coil can be either flat or three dimensional (cylindrical, cubic, etc.).

All internal components may be either housed inside a rigid housing (for example a cylindrical glass tube or a coin-shape container) or potted together in order to protect them from the implant environment. The housing can be made from a variety of materials such as glass, ceramics, polymers, silicone, Parylene, etc, and can be attached to the substrate using any method known in the art. In the embodiment where the elements are just potted together, the potting/coating material and its method of application can be any biocompatible material known to those skilled in the art. The substrate (with the interfaces) may be connected to the housing using either a hermetic sealing approach or any other connection known in the arts.

In some cases an extra layer of material (can be organic, or metal, or any biocompatible material) can be applied to parts of the implantable platform, in order to protect various parts of the system. Examples of coatings are polymers, Parylene, silicone, hydrogels, titanium, nitrides, oxides, carbides, silicides, etc. For example the coating can be applied to parts of the substrate and/or housing, leaving only the interfaces (or parts of the interfaces) open. This coating can be any biocompatible material or multiple layers of various biocompatible materials. Coatings on various parts of the system may also be composed of different materials, different thicknesses, etc. Based on specific applications, a coating may also be applied to parts of the modular element (device) that is connected to the platform. This coating can be either similar or different than the coating applied to the rest of the system.

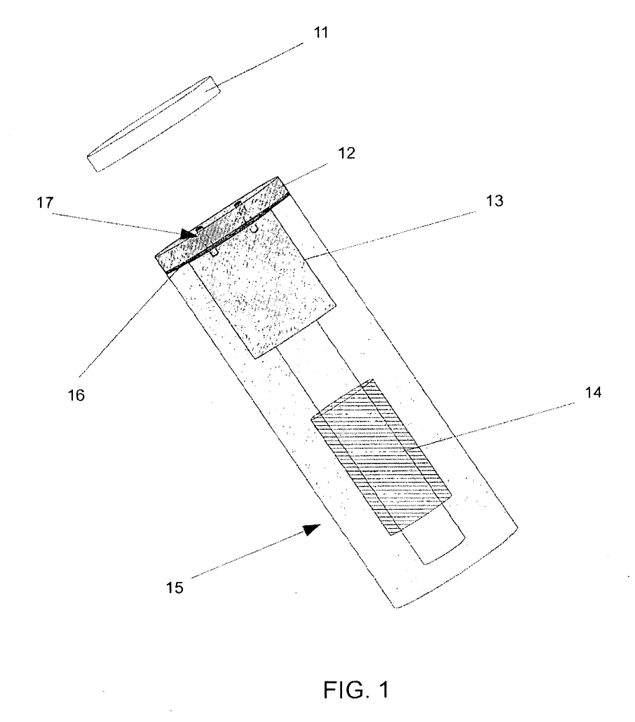

FIG. 1 shows a functional element 11, which may be attached separately to a housing 15. The functional element 11 is connected through a substrate 12 to an electronic component 13. Hermetic feed-through contacts 17 or wiring is used to provide the connection between the functional element 11 and the electronic component 13. The electronic component 13 is connected to a power source 14 which also integrates wireless communications capability. The power source 14 is designed to wirelessly communicate with an external readout unit (not shown). The external readout unit may also provide power to the power source 14 via magnetic telemetry or another method known in the art.

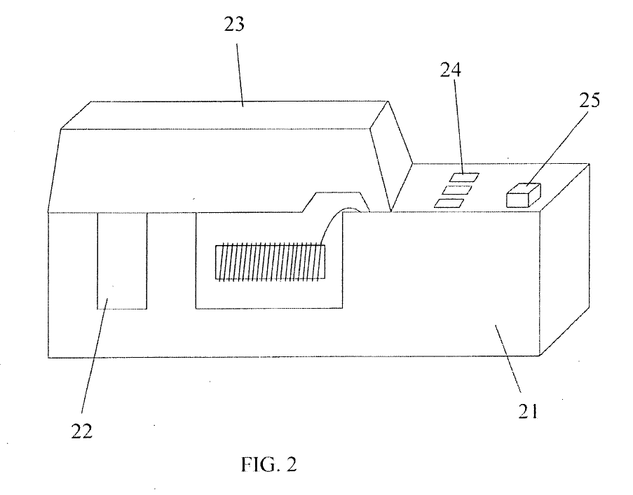

FIG. 2 shows another embodiment of an implantable wireless platform, in which a substrate 21 contains cavities 22 such that system components (such as an electronic component, coils, batteries, getters, etc) can be placed into these cavities 22, and a cap 23 can be attached to the substrate 21 such that it seals these components from the environment. In some embodiments, the substrate 21 or the cap 23 or both may have multiple cavities 22 (various depths, shapes, etc.) in order to accommodate the various components. The substrate 21 and the cap 23 can be made from any similar or dissimilar material (such as silicon, glass, ceramics, carbides, alloys, metals, hard polymers, Teflon, etc), and the cavities 22 can be formed by any method known in the art, including but not limited to machining, micromachining, potting, stamping, pre-forming, milling, wet etching, dry etching, sand blasting, water jet cutting, drilling, etc. The cap 23 can be attached to the substrate using any attachment method suitable for the intended use of the platform. The attachment method can either be hermetic or non-hermetic. Examples of attachment methods include (but are not limited to) anodic bonding, glass frit bonding, fusion bonding, plasma assisted bonding, epoxy bonding, adhesive bonding, welding, compression bonding, thermal compression bonding, metal-metal bonds, etc.

FIG. 2 also shows that the substrate 21 will also contain interfaces 24 for one or more functional elements 25 to be connected to this wireless platform. These interfaces 24 may include but are not limited to electrical contacts, fluid contacts/interface, optical interfaces, magnetic interface, capacitive interface, or a combination thereof. The interfaces 24 may be either fabricated as part of, or placed on the substrate 21, such that there will be connections from the interfaces 24 to the remaining components of the implantable system. The interfaces 24 may be two-dimensional (i.e. pads, electric contacts, etc.), or three-dimensional (i.e. tubes, etc.), or any combination or shape that is useful for the specific application. The interfaces 24 would usually be on one side of the substrate 21, and feedthroughs (preferably hermetic, but not necessary, based on the application) will be used to connect the interfaces 24 to the other components of the system inside the sealed cavities.

These components may include an electronic component, inductor coil, charge storing device, batteries, or any other kinds of components (based on the application). The inductor coil can be made using any method known in the art, such as winding a conductor around a ferrite core (air cores are possible as well), depositing (electroplating, sputtering, evaporating, screen printing, etc), a conductive coil (preferably made from a highly conductive metal such as silver, copper, gold, etc.), on a substrate (rigid or flexible), or any other method known to those skilled in the art. The inductor coil can be either flat or three dimensional (cylindrical, cubic, etc.).

In some cases an extra layer of material (can be organic, or metal, or any biocompatible material) can be applied to parts of the implantable platform, in order to protect various parts of the system. Examples of coatings are polymers, Parylene, silicone, hydrogels, titanium, nitrides, oxides, carbides, silicides, etc. For example the coating can be applied to parts of the substrate and/or housing, leaving only the interfaces (or parts of the interfaces) open. This coating can be any biocompatible material or multiple layers of various biocompatible materials. Coatings on various parts of the system may also be composed of different materials, different thicknesses, etc. Based on specific applications, a coating may also be applied to parts of the modular element (device) that is connected to the platform. This coating can be either similar or different than the coating applied to the rest of the system.

Another preferred embodiment for the wireless implantable platform uses a more flexible approach for platform where instead of one self-contained unit, the platform is formed using multiple subassemblies that are attached together. For example, the substrate 21 containing the connection interfaces 24, along with some other components (such as an electronic component) can be placed together and form one subassembly, and other system components (such as additional electronic components, inductor coil, batteries, etc), form a second subassembly. The two subassemblies will be connected together to form a wireless implantable platform. This approach allows a more flexible platform which allows parts of the implantable platform to be placed inside certain parts of the body, while other parts of the platform can be placed elsewhere, either inside or outside the body. One of the main advantages of this approach is that it allows the smallest possible footprint for the implanted portion in places where space is critical, and allows the larger/bulkier portions to be placed in a separate (and possibly more favorable) location.

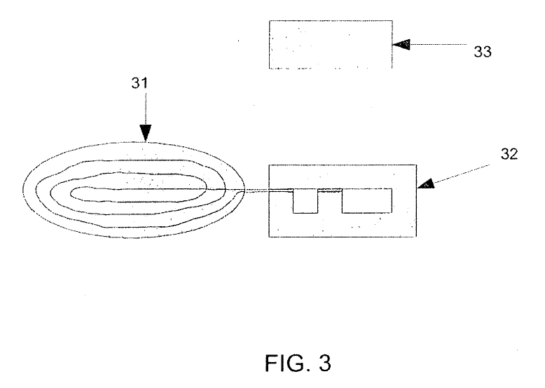

In FIG. 3, a power and wireless communications unit 31 is connected to an interface unit 32 which is connected to a functional element 33. In this embodiment, one or more interfaces 32 along with possibly some other components such as an electronic component can be either placed (i.e. mounted, or otherwise attached) on a substrate or be potted together in order to form one subassembly. As in the previous embodiments the interfaces 32 will be such that one or more devices with specific functionalities can be attached via the interface 32 to the wireless implantable platform. These interfaces 32 may include but are not limited to electrical contacts, fluid contacts/interface, optical interfaces, magnetic interface, capacitive interface, or a combination thereof The interfaces 32 may be either fabricated as part of, or placed on a substrate, such that there will be connections from the interface 32 to the remaining components of the implantable system. The interfaces 32 may be two-dimensional (i.e. pads, electric contacts, etc.), or three-dimensional (i.e. tubes, etc.), or any combination or shape that is useful for the specific application.

The substrate on which the interfaces 32 are placed or fabricated on, can be either rigid or flexible, or it can be partly rigid and partly flexible (for example rigid-flex substrates, where part of the substrate is rigid and the other part is flexible). In the case of flexible substrates, various polymers, Parylene, silicone, or other biocompatible flexible material may be used. In the case of rigid substrates, glass, silicon, ceramics, carbides, alloys, metals, hard polymers, Teflon, are some examples of substrate materials, although other types of materials can also be used for the substrate. In the case of rigid-flex substrates, the rigid and flexible parts may be made from dissimilar material.

In another embodiment, interfaces and possibly an electronic component may be connected together using various methods known in the art (for example wirebonding, flexible connectors, etc.), and instead of the components being mounted on a substrate, they can just be potted together using biocompatible epoxy or any other potting material.

This subassembly can be connected to a second subassembly consisting of other components such as other electronic components, an inductor coil, possibly a battery, and a charge storing device (such as capacitor or rechargeable battery).

The inductor coil can be made using any method known in the art, such as winding a conductor (preferably made from a highly conductive metal such as silver, copper, gold, etc.) around a ferrite core, depositing (electroplating, sputtering, evaporating, screen printing, etc), a conductive coil (preferably made from a highly conductive metal such as silver, copper, gold, etc.), on a substrate (rigid or flexible), or any other method known to those skilled in the art. Depending on implant location as well as factors such as readout distance, coil positioning, anchoring, etc., a wide variety of coil/core configurations can be used for the inductor coil (i.e. flat coil or three dimensional (cylindrical, cubic, or any other type of coil configuration). One advantage to using a flat coil configuration is that it can be easily implanted in certain areas where a large bulky 3-dimensional configuration is not suitable.

The wireless platform may include a battery (either rechargeable or not). The electronic component may include an ASIC, a diode, a capacitor, or other electronic circuitry.

Components in each subassembly may be either mounted on a substrate as mentioned above or potted together, or a combination of both. For example, the coil, and other electronic components can be either housed together (for example in a flat housing structure) or mounted on a substrate, or potted together or a combination thereof. The housing can be made from a variety of materials such as glass, ceramics, polymers, etc. In the embodiment where the elements are just potted together, the potting/coating material and its method of application can be any biocompatible material known to those skilled in the art.

In some cases an extra layer of material (can be organic, or metal, or any biocompatible material) can be applied to parts of the implantable platform, in order to protect various parts of the platform. For example the coating can be applied to parts of the subassembly, leaving only parts of the interfaces to be exposed to the environment. This coating can be any biocompatible material or multiple layers of various biocompatible materials (examples are polymers, Parylene, silicone, hydrogels, titanium, nitrides, oxides, carbides, silicides, etc).

Based on specific applications, a coating may also be applied to parts of the modular element (device) that is connected to the platform. This coating can be either similar or different than the coating applied to the rest of the system.

In all cases, the two subassemblies need to be connected to each other in order to provide electrical connection between the components. The connection can be either a flexible connection or a rigid connection or a connection which has a rigid part as well as a flexible part. If desired, the connecting portion can be coated, or potted or covered with a biocompatible material.

For all the embodiments discussed above, one preferred communication/tele-powering scheme for the implantable wireless platform is based on magnetic telemetry. Without an external reader present, the implant device lays passive and without any internal means to power itself When stimulation is desired, the external unit is brought into a suitable range to the implant. The external unit then creates an RF (Radio Frequency) magnetic field large enough to induce sufficient voltage across the implant coil. When such a sufficient voltage exists across the implant coil, the implant circuit may rectify the alternating waveform to create a direct voltage, which analog and/or digital circuitry may use as a power supply. At this point the implant can be considered alert and start stimulation, in the preferred embodiment, also ready for commands from the reader. As an alternative, in addition to the wireless powering there may be a combination of other powering and/or charging devices including but not limited to a battery or batteries, rechargeable battery or batteries, one or more capacitors, and one or more super capacitors.

As those skilled in magnetic telemetry are aware, a number of modulation schemes are available for transmitting data via magnetic coupling. The preferred schemes include but are not limited to amplitude modulation, frequency modulation, frequency shift keying, phase shift keying, and also spread spectrum techniques. The preferred modulation scheme may be determined by the specifications of an individual application, and is not intended to be limited under this invention.

In addition to the many available modulation techniques are the many technologies developed that allow the implant to communicate back to the reader the signal containing other information such as pressure, flow, Ph, CO2, neuron activities.

It is understood that the reader device may transmit either a continuous level of RF power to supply the implant's needed energy, or it may pulse the power allowing temporary storage in a battery or capacitor device. Similarly, the implant may signal back to the reader at any interval in time, delayed or instantaneous, during reader RF transmission or alternately in the absence of reader transmission. The implant may include a single coil antenna for both reception and transmission, or it may include two antennas, one each for transmission and reception.

The readout device may include an inductor for communicating with and powering the implant via magnetic telemetry. Also included is signal reception, signal processing, and transmission circuitry for data analysis and subsequent communication. There are many techniques for construction of the reader coil and processing electronic components known to those skilled in the art. The reader may interface to a display, computer, or other data logging device. In a preferred embodiment, external electronics consist of a readout unit and at least one antenna and the readout unit will receive data from the implant using the 13.56 MHz ISM band. Two modes of operation will be possible: (1) a data-logging measurement mode with optional data rates from 1 Hz and below, and (2) a real-time dynamic measurement mode with data rates from 100 to 500 Hz, for compliance and impulse tests. The readout unit can be comprised of an analog RF front end, a receiver/demodulator, and a digital processor plus user interface. The graphical user interface program used to control information (e.g., ICP monitor) can be created in Labview and C.

The implantable wireless platform may also need an anchoring mechanism in order to avoid movement of the implant components. Anchoring provisions may be incorporated directly into the platform (for example part of the housing) or may alternatively be added with an additional assembly step. An example of this would be insertion of the implantable part of the platform into a molded plastic or metal shell that incorporates anchoring provisions. Many such packaging schemes are known to those familiar with the art, and the present description should not be construed as limiting.

The anchoring mechanism can be any type of anchor known in the art, for example the implantable unit can be attached to the skull or scalp using wires, screws (helical or otherwise), bolts, a mesh, stents, springs, stitches, a tine that expands, etc. The anchoring mechanism can also be part of another device. The anchoring mechanisms can be made from one or more of the following materials, but not limited to, Nitinol, Teflon, Parylene, Polymer, or Metal.

Examples of devices that can be attached to the wireless implantable platform are sensors, actuators, other devices that provide structural functions, or combinations of these devices.

Various examples of miniature sensors are known to those skilled in the art, and any one or more of these suitable sensors can be utilized in the wireless platform. While the specific type of sensor(s) chosen will depend on the application of the implantable wireless platform, the sensor(s) should be of a sufficiently small size in order to facilitate placement and implantation. Additionally multiple sensors with different functionalities can also be incorporated into the wireless platform. Various types of sensors include but are not limited to: pressure sensors, temperature sensors, flow sensors, velocity sensors, vibration sensors, acceleration sensors, gas content (e.g., O2 and CO2), and chemical sensors.

Examples of actuating devices that can be attached to the wireless implantable platform include but are not limited to: deep brain stimulating probes, cardiac stimulators, thermal generators, voltage sources, current sources, probes, electrodes, drug delivery pumps, valves, meters, microtools for localized surgical procedures, radiation emitting sources, defibrillators, muscle stimulators, and pacing stimulators.

Examples of devices that can be attached to the wireless implantable platform for providing structural functions include but are not limited to: anchoring devices, attachment devices, and devices for enhancement of mechanical strength.

These implantable devices can significantly improve the tailored treatment of many severe diseases through: non-invasive stimulating/monitoring, real time, detailed, easy to use, low cost, home monitoring/stimulation capability, chronic use.

These implantable devices can be used for different internal organs including but not limited to one or more of the following organs: heart, brain, kidney, lung, bladder, reproductive systems, abdomen.

In the wireless system, the external readout unit is capable of performing one or more of the following: remote monitoring of patients, including but not limited to home monitoring; monitoring of patients with telephone-based (or similar method) data and information delivery; monitoring of patients with wireless telephone-based (or similar method) data and information delivery; monitoring of patients with web-based (or similar method) data and information delivery; closed-loop functions (such as but not limited to, drug delivery and neuro stimulation) to treat diseases; warning systems for critical worsening of diseases and related conditions; portable or ambulatory monitoring or diagnostic systems; battery-operation capability; data storage; reporting global positioning coordinates for emergency applications; communication with other medical devices including but not limited to pacemakers, defibrillator, implantable cardioverter defibrillator, implantable drug delivery systems, non-implantable drug delivery systems, neuro stimulators, and wireless medical management systems.

The devices may work continuously or it may do so periodically. In a periodic operation, one embodiment includes one phase for charging a charge storing device (such as a capacitor or rechargeable battery, or a combination therefore) as part of the implant and another phase for operation (e.g., sensing and/or stimulation). The charging and operation phases may overlap.

In all embodiments, one or more implantable devices may be used, either in close proximity, or in separate locations in the one or more body organs. In some cases, multiple sensing and/or stimulating elements on either the same substrate or multiple substrates can be used in order to stimulate various parts of one or more organs.

For all of the embodiments discussed, additional components such as batteries, various electronic components, multiple coils, multiple sensors, sensor/actuator combinations can be incorporated into the entire system. Batteries include rechargeable batteries.

In some cases, the multiple devices may share a common coil, or other common system elements. In other cases, the multiple sensing devices may each be completely separate units and not share any common elements.

In addition to medical applications, the wireless platform can also be used for non-medical applications such as aerospace, automotive, defense, bio-hazard detection, and various other industrial applications.

There are many reasons that the inventors feel the invention is not obvious to one skilled in the art. Examples include, but are not limited to, the wide variability in functional elements (e.g. size, functionality, power requirements, interface requirements), the difficulty in manufacturing to the tolerances required for interchangeability, different connection requirements for multiple subassemblies, different power requirements for multiple subassemblies, different connection requirements for the power sources discussed, and the difficulty in standardizing power supplies capable of meeting the requirements of the various modular wireless implantable systems that have been considered.

The foregoing disclosure includes the best mode devised by the inventors for practicing the invention. It is apparent, however, that several variations in the apparatuses and methods of the present invention may be conceivable by one skilled in the art. Inasmuch as the foregoing disclosure is intended to enable one skilled in the pertinent art to practice the instant invention, it should not be construed to be limited thereby, but should be construed to include such aforementioned variations.

1. A modular system for a wireless implantable device which is capable of at least one function, wherein the modular system has one or more subassemblies, the modular system comprising:
at least one functional unit designed to measure or affect a parameter of a patient's body wherein the functional unit is interchangeable with one or more functional units capable of measuring or affecting different body parameters;
an electronic component capable of interpreting the measurement of the functional unit or actuating the functional unit;
a wireless communications unit capable of sending and receiving data from a remote control and storage device;
a power supply capable of providing power to the functional unit, electronics, and wireless communications unit, wherein the power supplied may be continuous or non-continuous;
a biocompatible housing for the functional unit, electronic component, wireless communications unit, and power supply; and
a non-implantable readout device, the readout device having telemetric means for at least one of electromagnetic telecommunication and electromagnetic wireless powering of other portions of the modular system.

2. The modular system of claim 1 wherein the functional unit is either a sensor designed to measure a body parameter or an actuator designed to affect a body parameter.

3. The modular system of claim 2 wherein the power supply is a battery.

4. The modular system of claim 2 wherein the power supply comprises a magnetic telemetry device capable of receiving and using power via magnetic telemetry from a device external to the patient's body.

5. The modular system of claim 2 wherein the functional unit is physically separate from the electronic component, wireless communications unit, and power supply.

6. The modular system of claim 5 wherein the power supply is a battery.

7. The modular system of claim 5 wherein the power supply comprises a magnetic telemetry device capable of receiving and using power via magnetic telemetry from a device external to the patient's body.

8. The modular system of claim 1 further including a programmable memory located within the biocompatible housing.

9. The modular system of claim 8 wherein the functional unit is either a sensor designed to measure a body parameter or an actuator designed to affect a body parameter.

10. The modular system of claim 9 wherein the power supply is a battery.

11. The modular system of claim 9 wherein the power supply comprises a magnetic telemetry device capable of receiving and using power via magnetic telemetry from a device external to the patient's body.

12. The modular system of claim 9 wherein the functional unit is physically separate from the electronic component, wireless communications unit, and power supply.

13. The modular system of claim 12 wherein the power supply is a battery.

14. The modular system of claim 12 wherein the power supply comprises a magnetic telemetry device capable of receiving and using power via magnetic telemetry from a device external to the patient's body.

15. The modular system of claim 1 which further includes an anchor device, integrated with the biocompatible housing, designed to prevent movement of the modular system within the patient's body.

16. The modular system of claim 15 wherein the functional unit is either a sensor designed to measure a body parameter or an actuator designed to affect a body parameter.

17. The modular system of claim 16 wherein the power supply is a battery.

18. The modular system of claim 16 wherein the power supply comprises a magnetic telemetry device capable of receiving and using power via magnetic telemetry from a device external to the patient's body.

19. The modular system of claim 16 wherein the functional unit is physically separate from the electronic component, wireless communications unit, and power supply.

20. The modular system of claim 19 wherein the power supply comprises a magnetic telemetry device capable of receiving and using power via magnetic telemetry from a device external to the patient's body.
